(19) United States
(12) Patent Application Publication
(10) Pub. No.: US 2024/0132870 A1
(43) Pub. Date: Apr. 25, 2024

(54) curing leukemia and easy births

(71) Applicant: Menni Menashe Zinger, Ashkelon (IL)

(72) Inventor: Menni Menashe Zinger, Ashkelon (IL)

(21) Appl. No.: 17/971,681

(22) Filed: Oct. 23, 2022

(51) Int. Cl. C12N 13/00; A61B 10/02

(52) U.S. Cl. CPC C12N 13/00; A61B 10/025; A61B 2010/0258

(57) ABSTRACT

At this invention I presents sophisticated way to treat leukemia and sophisticated way to have births.

With using quantum mechanics and inspiration zones and model with example of bone marrow of healthy person who is relative of a patient or genetically close to the patient and by duplicating/multiplying bone marrow of the healthy relative, genetically close person by transmit the magnetic/electro magnetic/electric field/t.v. waves, frequency/radio waves, frequency on a plate near the sample of bone marrow plate by receiving the transmission with using receiver/scanner and passing it with using antenna/cables to transmitter/projector of magnetic/electro magnetic/electric field/t.v. waves, frequency/radio waves, frequency on the empty plate a duplicated sample should appear from thin air on the empty plate/s.

By using time machines and quantum mechanics and inspiration zones and model with sample of bone marrow that was taken from a patient from time he/she were healthy, without leukemia, 10 years, 20 years, 30 years before, at past the patient can cure itself in case there are no relatives to support him and to donate him/her a sample of bone marrow and the healthy sample beyond time can be duplicated/multiplied as much as needed and can be used to cure the patient by using transmit the magnetic/electro magnetic/electric field/t.v. waves, frequency/radio waves, frequency on a plate near the sample of bone marrow plate by receiving the transmission with using receiver/scanner and passing it with using antenna/cables to transmitter/projector of magnetic/electro magnetic/electric field/t.v. waves, frequency/radio waves, frequency on the empty plate a duplicated sample should appear from thin air on the empty plate/s.

By using quantum mechanics and inspiration zones a woman can have a birth easily without pains, suffering and danger to herself and to the baby at short time at time she is ready and not only at the last minute, even few days before at 9 mount of pregnancy by launching the baby/embryo using quantum mechanics and inspiration zones and passing the baby from 100% into 0% and from 0% into 100% again on the bad for baby at no time, zero time, alive and well, see my inventions 266246.il.2019. and my invention 17080105 u.s. near bad for baby ready for him/her when his/hers mother is at the same room or next room, ready to welcome him/hers into the world.

1. using quantum mechanics band inspiration zones and a model with sample of bone marrow of healthy person who is a relative, genetically close person to the patient and duplicating the sample of bone marrow and by duplicating/multiplying bone marrow of the healthy relative, genetically close person by transmit the magnetic/electro magnetic/electric field/t.v. waves, frequency/radio waves, frequency on a plate near the sample of bone marrow plate by receiving the transmission with using receiver/scanner and passing it with using antenna/cables to transmitter/projector of magnetic/electro magnetic/electric field/t.v. waves, frequency/radio waves, frequency on the empty plate a duplicated sample should appear from thin air on the empty plate/s. see my invention 266246.il.2019. and my invention 17080105 u.s.

2. using quantum mechanics and inspiration zones and time machines in order to take a sample from a patient before he/she got ill with leukemia and make it possible for the patient to save himself/herself with self donation from time he/she were healthy in case there is no relative who can donate or in case it is better to the patient to do so in order to recover on leukemia and duplicating the healthy sample/multiplying it as needed to empty plate/s by transmit the magnetic/electro magnetic/electric field/t.v. waves, frequency/radio waves, frequency on a plate near the sample of bone marrow plate by receiving the transmission with using receiver/scanner and passing it with using antenna/cables to transmitter/projector of magnetic/electro magnetic/electric field/t.v. waves, frequency/radio waves, frequency on the empty plate a duplicated sample should appear from thin air on the empty plate/s. see my invention 266246.il.2019. and my invention 17080105 u.s.

3. using quantum and inspiration zones in order to launch an embryo/baby from his/hers mother body to near by bad for baby by diluting him from 100% into 0% and crystalizing the baby, thicken him/her on the baby bad back into 100% at no time, zero time in order to not cause sorrow and pain and damage to the mother and babies because of the berth with is at great effort and pressures, see my invention 266246.il.2019. and my invention 17080105 u.s.
